(19) United States
(12) Patent Application Publication
(10) Pub. No.: US 2011/0275836 A1
(43) Pub. Date: Nov. 10, 2011

(54) PROCESS FOR THE PREPARATION OF DULOXETINE AND SALTS THEREOF

(75) Inventors: Ravi Ponnaiah, Vadodara (IN); Ashok Prasad, Vadodara (IN); Killol Patel, Vadodara (IN); Hitarth Harshendu Acharya, Vadodara (IN); Vilas Katore, Vadodara (IN); Bhavik Shah, Vadodara (IN)

(73) Assignee: ALEMBIC PHARMACEUTICALS LIMITED, VADODARA (IN)

(21) Appl. No.: 13/143,110

(22) PCT Filed: Dec. 28, 2009
(86) PCT No.: PCT/IB2009/055958

§ 371 Date: Jul. 1, 2011

(30) Foreign Application Priority Data
Jan. 6, 2009 (IN) 31/MUM/2009

(51) Int. Cl. C07D 333/20

(52) U.S. Cl. 549/75

(57) ABSTRACT

The present invention relates to improved process for the preparation of Duloxetine of formula (I) and salts thereof wherein said improvement takes place in step of condensation.

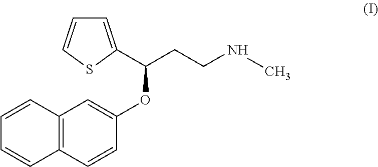

### FIELD OF THE INVENTION

The present invention relates to an improved process for the preparation of Duloxetine of formula (I) and salts thereof.

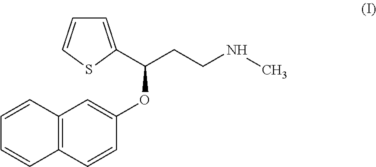

### BACKGROUND OF THE INVENTION

Duloxetine of formula (I) chemically known as (+)-(S)-N-methyl-γ-(1-naphthyloxy)-2-thiophenepropylamine belongs to class of antidepressant. Duloxetine is a serotonin-norepinephrine reuptake inhibitor (SNRI) used for major depressive disorder (MDD), generalized anxiety disorder (GAD), pain related to diabetic neuropathy and fibromyalgia Its hydrochloride salt is marketed under brand name of Cymbalta®.

Duloxetine and its pharmaceutically acceptable salt were first disclosed in U.S. Pat. No. 5,023,269. The process for synthesis of Racemic Duloxetine as described in this patent is as follows:

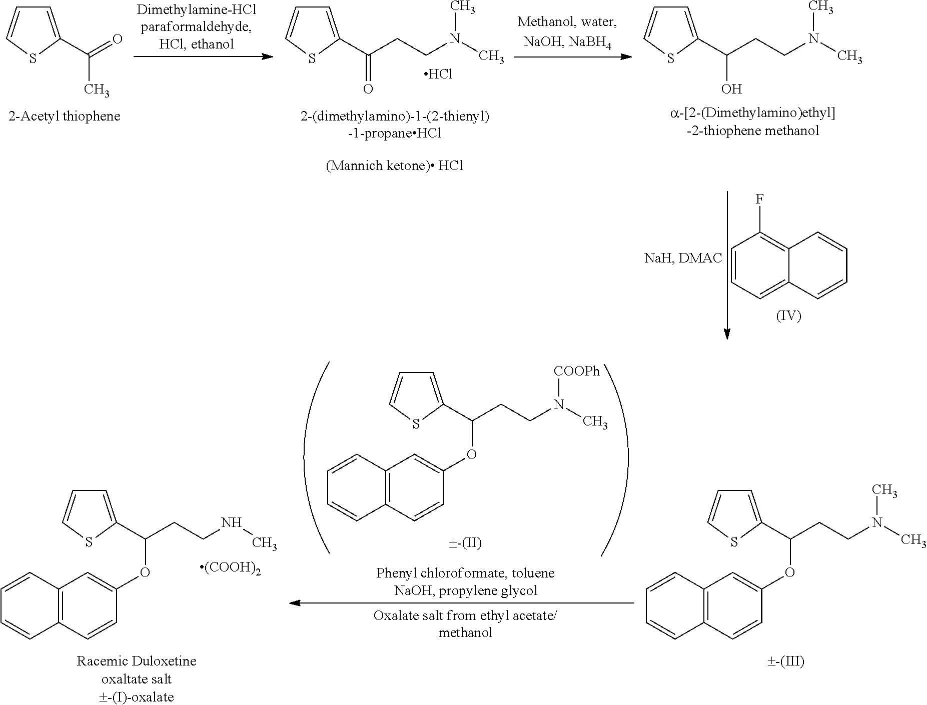

It is disclosed that compound of formula ±-(III) is obtained by condensation of corresponding racemic alcohol and compound of formula (IV) in the presence of alkali metal hydrides and suitable aprotic solvent.

EP 457559 and Tetrahedron Letters, Vol. 31, No. 49, pp 7101-7104 describe process for preparation of Duloxetine which involves condensation of chiral 13-hydroxy alcohol of formula (V) with compound of formula (IV) in the presence of Sodium hydride. However the reported reaction period is in range of 48 to 72 hours.

When a chiral β-hydroxy alcohol of formula (V) is reacted with compound of formula (IV) in aforesaid conditions the reaction time is longer which results into racemization of optically active product of formula (III).

Reinheimer, et al in their publications in Journal of American Chemical Society 1958, Vol. 80, page 164-168 and 1961 Vol. 83, page 2873-2877 have studied effect of added salts on rate of reaction of 2,4-dinitrochlorobenzene and alkali metal methoxides.

U.S. Pat. No. 5,362,886 relates to reaction of chiral β-hydroxy alcohol of formula (V) with compound of formula (IV) in the presence of sodium hydride and potassium compound chosen from potassium benzoate or acetate. The patent reports increase in the rate of reaction due to the presence of potassium benzoate or acetate.

The inventors of present invention have observed that the condensation of chiral 13-hydroxy alcohol of formula (V) with compound of formula (IV) in presence of sodium hydride is highly assisted in presence of catalytic amount of potassium iodide and the process efficiency is unexpectedly enhanced due to this. The advantage of potassium iodide is that it is cheaper in cost, easily available and operation effortless to handle during scale-up procedures.

### SUMMARY OF THE INVENTION

The present invention provides an improved process for preparation of Duloxetine of formula (I) and salts thereof.

The present invention provides a process for the preparation of compound of formula (III) or salts thereof, comprising of condensing compound of formula (V) with compound of formula (IV) in the presence of sodium hydride characterized in that the said condensation is carried out in the presence of catalytic amount of potassium iodide and optionally converting it to salt thereof.

Another object of the present invention is to provide a process for the preparation of Duloxetine of formula (I) and salts thereof comprising steps of:
(a) reacting compound of formula (VII) with dimethyl amine and paraformaldehyde to obtain compound of formula (VI)
(b) reducing compound of formula (VI) and resolving to obtain compound of formula (V)
(c) condensing compound of formula (V) with compound of formula (IV) in the presence of sodium hydride characterized in that the said condensation is carried out in the presence of catalytic amount of potassium iodide to obtain compound of formula (III) and optionally converting it to salts thereof
(d) reacting compound of formula (III) with phenylchloroformate to obtain compound of formula (II)
(e) converting compound of formula (II) to compound of formula (I) or salt thereof.

Yet another object of present invention is to provide process of preparation of compound of formula (I) or salts thereof comprising a step of condensing compound of formula (V) with compound of formula (IV) in the presence of sodium hydride characterized in that the said condensation is carried out in the presence of catalytic amount of potassium iodide to obtain compound of formula (III) and optionally converting it to salts thereof.

### DETAILED DESCRIPTION OF THE INVENTION

The present invention provides a process for the preparation of compound of formula (I) or salts thereof comprising a step of condensing compound of formula (V) with compound of formula (IV) in the presence of sodium hydride characterized in that the said condensation is carried out in the presence of catalytic amount of potassium iodide to obtain compound of formula (III) and optionally converting it to salts thereof.

Another embodiment of the present invention provides a process for the preparation of Duloxetine of formula (I) and salts thereof comprising steps of:
(a) reacting compound of formula (VII) with dimethyl amine and paraformaldehyde to obtain compound of formula (VI)
(b) reducing compound of formula (VI) and resolving to obtain compound of formula (V)
(c) condensing compound of formula (V) with compound of formula (IV) in the presence of sodium hydride characterized in that the said condensation is carried out in the presence of catalytic amount of potassium iodide to obtain compound of formula (III) and optionally converting it to salt thereof
(d) reacting compound of formula (III) with phenylchloroformate to obtain compound of formula (II)
(e) converting compound of formula (II) to compound of formula (I) or salts thereof.

Yet another embodiment of the present invention provides a process for the preparation of compound of formula (III) or salts thereof, comprising of condensing compound of formula (V) with compound of formula (IV) in the presence of sodium hydride characterized in that the said condensation is carried out in the presence of catalytic amount of potassium iodide and optionally converting it to salt thereof.

The Schematic representation of present invention is as shown in Scheme-I

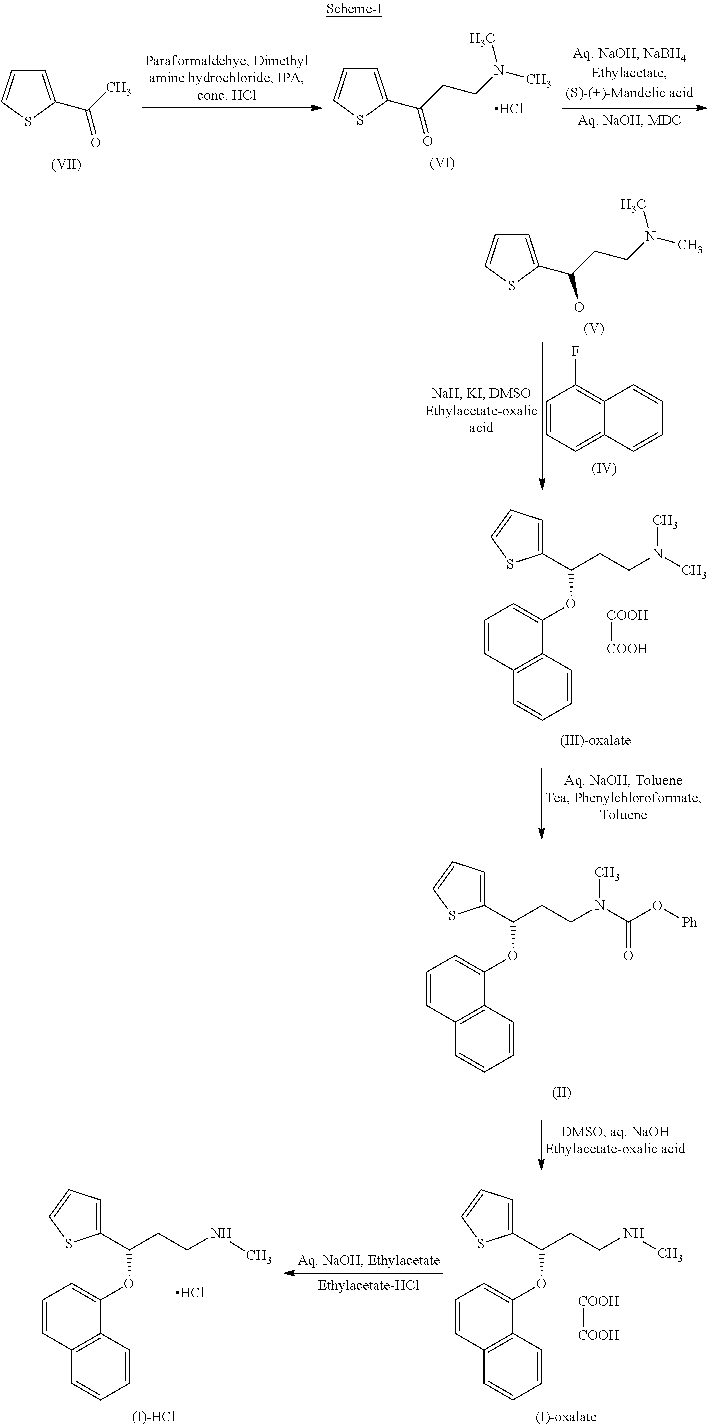

Compound of formula (III) can be prepared by any method known perse or by process known in the art.

In a preferred embodiment step (a) comprises of reacting compound of formula (VII) with dimethyl amine and paraformaldehyde to obtain compound of formula (VI) in presence of concentrated hydrochloric acid in isopropyl alcohol as solvent.

Step (b) comprises of reducing compound of formula (VI) in presence of aqueous sodium hydroxide and further resolving it using mandelic acid in ethyl acetate to obtain compound of formula (V).

Step (c) comprises condensation of compound of formula (V) with compound of formula (IV) in the presence of sodium hydride and catalytic amount of KI in dimethylsulphoxide as solvent to produce compound of formula (III). The reaction is carried out in the temperature range of about 25° to about reflux temperature of the solvent, preferably at about 50° C. to about 90° C. The molar equivalence of KI with respect to compound of formula (V) is about 0.05 to 0.5 mole ratio.

The preferred solvent for the step of condensation is dimethylsulphoxide. However, a person skilled in the art may use any obvious variant of solvents known for the step of condensation of compound of formula (V) and (IV).

Compound of formula (III) can be optionally converted to its oxalate salt in presence of ethyl acetate.

Further compound of formula (III) is reacted with phenylchloroformate in presence of triethylamine as base and toluene as solvent to obtain compound of formula (II) which is a carbamate ester.

The carbamate ester of formula (II) is hydrolyzed in the presence of aqueous sodium hydroxide in dimethylsulphoxide as solvent, to obtain compound of formula (I).

The compound of formula (I) can be optionally converted to its pharmaceutically acceptable salts.

Said salts of Duloxetine of formula (I) includes but are not limited to organic and inorganic acid salts for example, hydrochloric, hydrobromic, sulfuric, phosphoric, para-toluenesulfonic, methanesulfonic, oxalic, maleic, acetic acid and the like.

The following examples illustrate the invention further. It should be understood however, that the invention is not confined to the specific limitations set forth in the individual example but rather to the scope of the appended claims.

### Example 1

### Preparation of 3-(N, N-Dimethylamino)-1-(2-thienyl) propan-1-one hydrochloride

Charge Isopropanol (250 ml) to the flask at 25-35° C. Charge Dimethyl amine hydrochloride (77.5 g) to the flask followed by Conc. HCl (4.0 ml) under stirring. Charge Paraformaldehyde (33.33 g) to the flask under stirring. Stir the reaction mass for 30 mins. Add 2-Acetyl thiophene (100.0 g) to the flask. Heat the reaction mixture and stir the reaction mixture at 70-75° C. After the completion of the reaction; cool the reaction mass. Filter the content. Wash the wet cake with Isopropanol. Suck dry the material. Dry the material in hot air oven.

### Example 2

### Preparation of 3-(dimethylamino)-1-(2-thienyl) propan-1-ol

Charge D M Water (500 ml) into a flask. Charge Sodium hydroxide (21.84 g) to the flask. Stir the reaction mass to get clear solution. Add 3-(N, N-Dimethylamino)-1-(2-thienyl) propan-1-one hydrochloride (100 g) to the flask under stirring. Cool the reaction mass to 10-15° C. Add Sodium borohydride (8.65 g) to the reaction mixture at 10-15° C. Stir the reaction mixture. Add Sodium borohydride (1.75 g) to the reaction mixture. Stir the reaction mixture. After the completion of the reaction; add Acetone (5.0 ml) to the reaction mixture. Stir for 30-45 min at 20-25° C. Filter the content at 20-25° C. Wash with D M water. Dry it at 50-55° C. in hot air oven.

### Stage-III: Preparation of S-(−)-3-(dimethylamino)-1-(2-thienyl) propan-1-ol

Charge fresh ethyl acetate (800 ml). Charge racemic alcohol (80 gm). Add S-(+)-Mandelic acid (39.5 g) to reaction mixture. Heat the reaction mixture to 45-50° C. Stir the reaction mixture at 45-50° C. Cool the reaction mixture to 25-30° C. Stir the reaction mixture at 25-30° C. Filter the content. Wash the wet cake with Ethyl acetate. Suck dry the wet cake. Dry the material in hot air oven at 40-50° C.

Charge D M Water (150 ml) to the flask. Charge Mandelate salt. Charge Dichloromethane (400 ml). Stir the reaction mass for 10 mins and then cool to 10-15° C. Add 1 N Sodium hydroxide solution (240 ml) to the reaction mixture. Stir the reaction mixture. Separate the org. layer. Re-extract the aq. layer with Dichloromethane (200 ml). Combine both org. layer and wash with D M Water. Remove Dichloromethane at 40-45° C. under vacuum. Add Cyclohexane (70 ml) to the reaction mass. Remove Cyclohexane at 40-45° C. under vacuum. Add Cyclohexane (100 ml) to the reaction mass. Cool the reaction mass to 10-15° C. Stir the reaction mixture. Filter the content. Wash the wet cake with chilled Cyclohexane. Suck dry the wet cake. Dry the material in hot air oven at 40-50° C.

### Stage-IV: Preparation of (3S)-N, N-dimethyl-3-(1-naphthyloxy)-3-(2-thienyl) propan-1-ammonium oxalate

Charge DMSO (500 ml) to the flask. Charge S-(−)-3-(dimethylamino)-1-(2-thienyl) propan-1-ol (100 g) to the flask. Stir the reaction mass for 10 mins to get clear solution. Cool to 10-15° C. Charge Sodium hydride (23.84 g) to the reaction mass at 10-15° C. Stir the reaction mixture for 30 min at ambient temperature. Add Potassium Iodide (9.0 g) at 20-25° C. Heat the reaction mixture to 65-75° C. Add a solution of 1-Fluoronaphthalene in DMSO (71.6 ml in 100 ml) to the reaction mixture. Stir the reaction mixture for 5 hrs. After the completion of the reaction, cool the reaction mixture to 20-25° C. Add methanol (5 ml) at 20-25° C. under nitrogen. Add D M Water (6000 ml) to the reaction mixture. Add Ethyl acetate (500 ml) to the reaction mixture. Stir the reaction mixture for 15 mins. Separate the org. layer. Re-extract the org. layer with Ethyl acetate (500 ml). Combine both org. layer and wash with D M Water (300 ml). Remove Ethyl acetate (˜500 ml) under vacuum at below 50° C. Add Ethyl acetate (500 ml) to the residual mass. Add Oxalic acid dihydrate (71.5 g) to the reaction mass. Add Methanol (50 ml) to the reaction mass. Stir the reaction mixture for 1 hr. Cool the reaction mixture to 0-5° C. Stir the reaction mixture for 2 hrs. at 0-5° C. Filter the content. Wash the wet cake with Ethyl acetate (100 ml). Suck dry the wet cake. Dry the wet material in hot air oven at 50-60° C.

### Stage-IV: Preparation of Duloxetine Hydrochloride

Charge D M Water (300 ml) to the flask. Charge (3S)-N, N-dimethyl-3-(1-naphthyloxy)-3-(2-thienyl) propan-1-ammonium oxalate (100 g) to the flask. Stir the reaction mixture for 10-15 mins. Add Toluene (500 ml) to the flask. Add 1N NaOH solution to the reaction mixture and adjust pH 9-10. Stir the reaction mixture for 30 mins. Separate the org. layer. Re-extract aq. layer with Toluene (500 ml). Combine both org. layer and wash with D M Water. Remove Toluene up to half volume under vacuum at below 50° C. Make up the volume up to 1000 ml with Toluene (˜500 ml) at 25-35° C. Add Triethylamine (6.95 ml) to the reaction mixture 25-35° C. Add Phenyl chloro formate (46.8 ml) to the reaction mixture 25-35° C. Heat the reaction mixture. Stir the reaction mixture for 1 hr at 60-65° C. Add Triethylamine (6.95 ml) to the reaction mixture. Stir the reaction mixture at 60-65° C. After the completion of the reaction; cool the reaction mixture to 20-25° C. Add 10% Sod. Carbonate soln to the reaction mixture. Stir the reaction mixture for 15-30 mins. Separate the org. layer. Wash the org. layer with D M Water. Remove Toluene completely under vacuum at below 50° C. Cool the residual mass to 25-35° C. Charge DMSO (500 ml) to the residual mass. Add 30% Sod. Hydroxide soln (150 ml) to reaction mixture. Heat the reaction mixture to 85-90° C. Stir the reaction mixture at 85-90° C. for 3 hrs. After the completion of the reaction; cool the reaction mass to 20-25° C. Add D M Water to the reaction mixture. Add Ethyl acetate to the reaction mixture. Stir the reaction mixture. Separate the org. layer. Re-extract the aq. layer with Ethyl acetate. Combine both org. layer and wash with D M Water. Remove Ethyl acetate completely under vacuum at below 50° C. Add Acetone (300 ml) to the residual mass at 20-25° C. Stir the reaction mixture for 10-15 mins. Add Ethyl acetate Hydrochloride (˜40 ml) to the reaction mixture to adjust pH 1-2. Stir the reaction mixture for 1 hrs at 20-25° C. Filter the content. Wash the wet cake with Acetone. Wash the wet cake with Ethyl acetate. Suck dry the wet cake. Dry the material under vacuum at 40-50° C.

1. A process for the preparation of compound of formula (I) or salts thereof comprising a step of condensing compound of formula (V) with compound of formula (IV) in the presence of sodium hydride characterized in that the said condensation is carried out in the presence of catalytic amount of potassium iodide to obtain compound of formula (III) and optionally converting it to salts thereof.

2. A process for the preparation of Duloxetine of formula (I) and salts thereof comprising steps of: 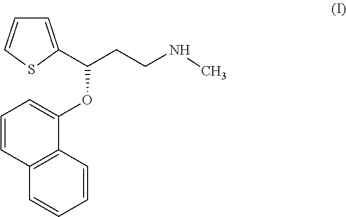
(a) reacting compound of formula (VII) with dimethyl amine and paraformaldehyde to obtain compound of formula (VI), 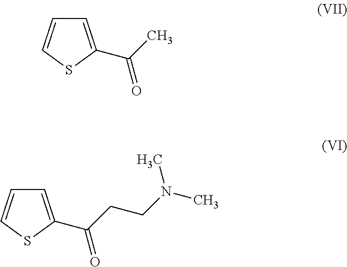
(b) reducing compound of formula (VI) and resolving to obtain compound of formula (v) 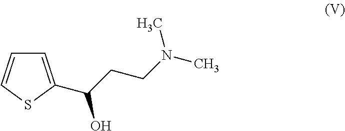
(c) condensing compound of formula (V) with compound of formula (IV) in the presence of sodium hydride characterized in that the said condensation is carried out in the presence of catalytic amount of potassium iodide to obtain compound of formula (III) and optionally converting it to salt thereof, 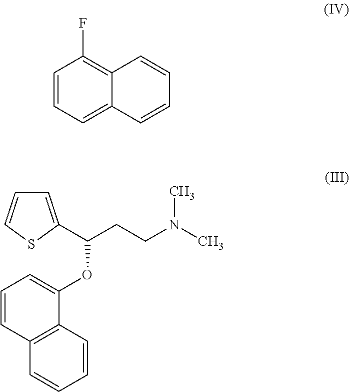
(d) reacting compound of formula (III) with phenylchloroformate to obtain compound of formula (II), 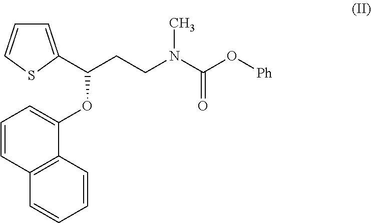
(e) converting compound of formula (II) to compound of formula (I) or salts thereof.

3. A process for the preparation of compound of formula (III) or salts thereof, comprising of condensing compound of formula (V) with compound of formula (IV) in the presence of sodium hydride characterized in that the said condensation is carried out in the presence of catalytic amount of potassium iodide and optionally converting it to salt thereof.

4. A process claimed in claim 1 wherein said condensation is carried out in the presence of dimethylsulphoxide as solvent.

5. A process claimed in claim 2 wherein said condensation in step (c) is carried out in the presence of dimethylsulphoxide as solvent.

6. A process claimed in claim 3 wherein said condensation is carried out in the presence of dimethylsulphoxide as solvent.
